(19) United States
(12) Patent Application Publication
(10) Pub. No.: US 2026/0076803 A1
(43) Pub. Date: Mar. 19, 2026

(54) POLYISOBUTYLENE-BASED POLYURETHANES FOR MEDICAL IMPLANT DEVICES

(71) Applicant: Innovia, Inc., Miami, FL (US)

(72) Inventor: Leonard Pinchuk, Miami, FL (US)

(73) Assignee: Innovia, Inc., Miami, FL (US)

(21) Appl. No.: 18/993,448

(22) PCT Filed: Jun. 23, 2023
(86) PCT No.: PCT/US2023/026110

§ 371 Date: Jan. 10, 2025

Related U.S. Application Data
(60) Provisional application No. 63/388,873, filed on Jul. 13, 2022.

(51) Int. Cl. A61F 2/38; A61L 27/18; C08G 18/12; C08G 18/40; C08G 18/44; C08G 18/48; C08G 18/62; C08G 18/76

(52) U.S. Cl. CPC A61F 2/3872; A61L 27/18; C08G 18/12; C08G 18/4063; C08G 18/44; C08G 18/4854; C08G 18/6204; C08G 18/7671

(57) ABSTRACT

Methods are disclosed for preparing polyurethane or polyurea polymers with crosslinks between polymer chains as well as orthopedic devices and other medical implant devices formed by the polyurethane or polyurea polymers and related methods of fabricating and/or assembling such devices. The crosslinks can enhance their mechanical, thermal, chemical, electrical, and dimensional properties, providing improved performance and expanding their range of applications compared to non-crosslinked polymers.

### CROSS-REFERENCE TO RELATED APPLICATIONS

The present disclosure claims priority from U.S. Provisional Appl. No. 63/388,873 filed on Jul. 13, 2023, herein incorporated by reference in its entirety.

### BACKGROUND

### 1. Field

The present disclosure relates to medical implant devices made for polymeric materials.

### 2. State of the Art

There is a need for tough long-lasting implantable dynamic and/or load-bearing prostheses such as heart valves and orthopedic devices, such as an artificial meniscus and/or anterior cruciate ligament (ACL), that can be implanted into humans to treat knee pain, damage, and/or instability.

In Mckeon, et al., “Preliminary Results From a US Clinical Trial of a Novel Synthetic Polymer Meniscal Implant,” First Published Sep. 29, 2020 Research Article Find in PubMed https://doi.org/10.1177/2325967120952414 The Orthopaedic Journal of Sports Medicine, 8(9), 2325967120952414 DOI: 10.1177/2325967120952414, the authors provide the need, the challenges, and a description of an artificial meniscus from NuSurface, which is probably the most advanced synthetic artificial meniscus made to date. The NuSurface meniscus is made from a polycarbonate-urethane (PCU) reinforced with high molecular weight polyethylene fibers. This PCU was patented for medical use by Leonard Pinchuk, the inventor on this patent application, in U.S. Pat. No. 5,133,742, L. Pinchuk, “Crack-Resistant Polycarbonate Urethane Polymer Prostheses,” 1992; and U.S. Pat. No. 5,229,431, L. Pinchuk, “Crack-Resistant Polycarbonate Urethane Polymer Prostheses,” 1993. Furthermore, this PCU is not biostable as summarized in L. Pinchuk, G. J. Wilson, J. J. Barry, R. T. Schoephoerster, J. M. Parel, J. P. Kennedy, Medical applications of poly(styrene-block-isobutylene-block-styrene) (“SIBS”), Biomaterials 29 (4) (2008) 448-460 doi:10.1016/j.Biomaterials.2007.09.041.

The PCU described by Mckeon et al. uses a polycarbonate diol shown schematically in FIG. 1. The polycarbonate diol can be formed by the condensation reaction of 1,6 hexanediol and ethylene carbonate(represented as HO-PC-OH in FIG. 1). The polycarbonate diol is reacted with a diisocyanate, such as 4,4′-Methylenebis(phenyl isocyanate) or MDI, to form a polycarbonate urethane prepolymer. The MDI is shown schematically in FIG. 2A. The polycarbonate urethane prepolymer is reacted with a chain extender 1,4-butanediol to form the PCU. The chain extender 1,4-butanediol is shown schematically in FIG. 2B, and the resultant PCU is shown on the bottom of FIG. 3.

Oxidation of the CH₂ groups in soft segments derived from the polycarbonate diol can occur in the body causing double bond formation and degradation as shown in FIG. 4. Such oxidation can cause embrittlement followed by cracking of the device and hydrolysis of the carbonate group. The aromatic hard segments derived from the MIDI and chain extender are more stable than the carbonate linkage due to resonance stabilization around the isocyanate group (also called a carbamate group).

The bottom line is that PCU is not a good material for a long-term orthopedic device as it can embrittle and degrade over time. Thus, there is a need for tougher long-lasting materials for medical implant devices, such as an artificial meniscus and/or anterior cruciate ligament.

### SUMMARY

The present disclosure describes methods for preparing polyurethane or polyurea polymers with crosslinks between polymer chains as well as orthopedic devices and other medical implant devices formed by the polyurethane or polyurea polymers and related methods of fabricating and/or assembling such devices. The crosslinks can enhance their mechanical, thermal, chemical, electrical, and dimensional properties, providing improved performance and expanding their range of applications compared to non-crosslinked polymers.

In one aspect, a method of forming a polyurethane or polyurea polymer is provided that involves reacting hydroxyl-terminated polyisobutylene with a diisocyanate to form a prepolymer. The prepolymer can be reacted with a trifunctional chain extender to form the polyurethane or polyurea polymer. The polyurethane or polyurea polymer in this aspect includes hard segments and soft segments with crosslinks between polymer chains in the hard segment of the polymer. The hard segments include at least one of a urethane, urea, or urethane urea derived from the diisocyanate. The soft segments include polyisobutylene derived from the hydroxyl-terminated polyisobutylene. The crosslinks between polymer chains in the polymer include crosslinks between hard segments in the polymer chains that are derived by reaction of the trifunctional chain extender and isocyanate groups of the diisocyanate.

In embodiments, at least the reaction of the prepolymer with the trifunctional chain extender can be carried out in a mold used to shape and form a medical implant device (e.g., orthopedic device or other medical implant device as described herein).

In another aspect, a method of forming a polyurethane or polyurea polymer is provided that involves synthesizing or obtaining hydroxyl-terminated polyisobutylene that includes a thermal-activated crosslinker. The hydroxyl-terminated polyisobutylene (with crosslinker) can be reacted with a diisocyanate to form a prepolymer. Heat can be applied to the prepolymer, or to a reaction product derived therefrom, to form the polyurethane or polyurea polymer. The polyurethane or polyurea polymer includes hard segments and soft segments with crosslinks between polymer chains in the soft segment of the polymer. The hard segments include at least one of a urethane, urea, or urethane urea derived from the diisocyanate. The soft segments include polyisobutylene derived from the hydroxyl-terminated polyisobutylene. The crosslinks between polymer chains in the polymer include crosslinks between soft segments in the polymer chains that are derived from the thermal-activated crosslinker.

In embodiments, at least the application of heat is carried out in a mold used to shape and form a medical implant device (e.g., orthopedic device or other medical implant device as described herein).

In embodiments, the prepolymer can be reacted with a trifunctional chain extender, and the heat is applied to the reaction product of the prepolymer and the trifunctional chain extender. In this case, the crosslinks between polymer chains in the polymer also include crosslinks between hard segments in the polymer chains that are derived by reaction of the trifunctional chain extender and isocyanate groups of the diisocyanate.

In embodiments, at least the reaction of the prepolymer with the trifunctional chain extender and the application of heat can be carried out in a mold used to shape and form a medical implant device (e.g., orthopedic device or other medical implant device as described herein).

In yet another aspect, a medical implant device is provided that includes a polyurethane or polyurea polymer including hard segments and soft segments with crosslinks between polymer chains in the polymer. The hard segments include at least one of a urethane, urea, or urethane urea derived from a diisocyanate. The soft segments include polyisobutylene derived from hydroxyl-terminated polyisobutylene.

In embodiments, the hard segments can be derived from a chain extender.

In embodiments, the crosslinks between polymer chains can be configured to link hard segments of the polymer chains.

In embodiments, the crosslinks between polymer chains can be configured to link soft segments of the polymer chains.

In embodiments, the crosslinks between polymer chains can be configured to link both hard segments and soft segments of the polymer chains.

In embodiments, the crosslinks between polymer chains can include crosslinks between hard segments in the polymer chains that are derived by reaction of a trifunctional chain extender and isocyanate groups of the diisocyanate.

In embodiments, the crosslinks between polymer chains can include crosslinks between soft segments in the polymer chains that are derived from a thermal-activated crosslinker.

In embodiments, the medical implant device can be an orthopedic implant device selected from the group consisting of an artificial meniscus, ACL, rotator cuff labrum, spinal disk, finger joint, impact dampening liner for artificial hip or knee prosthesis, and a soft tissue replacement.

In embodiments, the medical implant device can be selected from the group consisting of a synthetic heart valve, a vascular graft, a cardiac pacemaker lead, a defibrillator lead, a catheter, an implantable prosthesis, a cardiac assist device, an artificial organ, and a drug delivery device.

In yet another aspect, an artificial meniscus is provided that includes an inner core encapsulated by an outer shell. The inner core can be formed from a first polyurethane or polyurea polymer including hard segments and soft segments, where the hard segments include at least one of a urethane, urea, or urethane urea derived from a diisocyanate, and the soft segments include polyisobutylene derived from hydroxyl-terminated polyisobutylene. The outer shell can be formed from a second polyurethane or polyurea polymer including hard segments and soft segments, where hard segments include at least one of a urethane, urea, or urethane urea derived from a diisocyanate, and the soft segments include polyisobutylene derived from hydroxyl-terminated polyisobutylene. The first polyurethane or polyurea polymer of the inner core is softer than the second polyurethane or polyurea polymer of the outer shell.

In embodiments, at least one of the first polyurethane or polyurea polymer of the inner core and the second first polyurethane or polyurea polymer of the outer shell can include crosslinks between polymer chains.

In embodiments, the crosslinks between polymer chains can be configured to link hard segments of the polymer chains; and/or the crosslinks between polymer chains can be configured to link soft segments of the polymer chains; and/or the crosslinks between polymer chains can be configured to link both hard segments and soft segments of the polymer chains.

In embodiments, the crosslinks between polymer chains can include crosslinks between hard segments in the polymer chains that are derived by reaction of a trifunctional chain extender and isocyanate groups of the diisocyanate.

In embodiments, the crosslinks between polymer chains can include crosslinks between soft segments in the polymer chains that are derived from a thermal-activated crosslinker.

In one or more of the aspects, the trifunctional chain extender can be selected from the group consisting of 2-hydroxyethyl-1,3-propanediol, 1,2,3-Propanetriol (glycerin), 1,2,3-propanetriamine, 2-hydroxyethyl-1,4-butanediol, 2-hydroxypropyl-1,4-butanediol, 3-hydroxypropyl-1,5-pentanediol, 3-aminopropyl-1,5-pentanediamine, 4-hydroxybuty1-1,6-hexanediol, 3-hydroxybutyl-1,6-hexanediol, and the like or combinations thereof.

In one or more of the aspects, the thermal-activated crosslinker can include BCB, which can be derived from a polymer selected from the group consisting of 4-Vinylbenzocyclobutene (VBCB), 4-methylvinylbenzocyclobutene, 4-Vinylbenzocyclopropene, 4-Vinylbenzo-2-methylcyclobutene, 4-Vinylbenzo-2-ethylcyclobutene or combinations thereof.

In one or more of the aspects, the hydroxyl-terminated polyisobutylene can include hydroxyl-terminated polyisobutylene diol.

In one or more of the aspects, the hydroxyl-terminated polyisobutylene can include hydroxyl-terminated polyisobutylene diol in combination with a polytetramethylene glycol and/or a polycarbonate diol.

In one or more of the aspects, the diisocyanate can include MDI.

Methods of forming and assembling medical implant devices from polyisobutylene-based polyurethane or polyurea polymers with crosslinks are also described and claimed.

### BRIEF DESCRIPTION OF THE DRAWINGS

The foregoing will be apparent from the following more particular description of example embodiments of the invention, as illustrated in the accompanying drawings in which like reference characters refer to the same parts throughout different views. The drawings are not necessarily to scale, emphasis being placed on illustrating embodiments of the invention.

FIG. 1 is a schematic diagram of a polycarbonate diol used to synthesize a prior art polycarbonate-urethane (shown in FIG. 3).

FIGS. 2A and 2B are schematic diagrams of MIDI and chain extender 1,4-butanediol, respectively, which are used to synthesize the prior art polycarbonate-urethane (shown in FIG. 3).

FIG. 3 is the polycarbonate-urethane made from polycarbonate diol, MDI and chain extender 1,4-butanediol.

FIG. 4 shows the degradation of prior art polycarbonate soft segment to double bonds followed by cleavage.

FIG. 5 is a schematic diagram showing polyethylene with primary carbons (PC), polypropylene with tertiary carbons (TC), and polyisobutylene with quaternary carbons (QC).

FIG. 6 is a schematic diagram of an example method (reactions steps) to prepare (synthesize) a PIB-PU according to an embodiment of the present disclosure.

FIGS. 7 and 8 are schematic diagrams of example methods (reactions) to prepare (synthesize) hydroxyl-terminated polyisobutylene, which can be used to prepare (synthesize) a PIB-PU according to an embodiment of the present disclosure.

FIG. 9 is a schematic diagram of an example method (reaction) that uses the hydroxyl-terminated polyisobutylene of FIG. 8 reacted with 4,4′-methylenebis (phenyl isocyanate) to form a prepolymer.

FIG. 10 is the reaction product of FIG. 9 prepolymer with a trifunctional chain extender to prepare (synthesize) a PIB-PU according to an embodiment of the present disclosure.

FIG. 11A is a schematic diagram of 4-vinylbenzocyclobutene (VBCB), which can be used to prepare (synthesize) a PIB-PU according to an embodiment of the present disclosure.

FIG. 11B is a schematic diagram of an example hydroxyl-terminated polyisobutylene diol with a thermal-activated crosslinker, which can be used to prepare (synthesize) a PIB-PU according to an embodiment of the present disclosure.

FIG. 12 is a schematic diagram illustrating crosslinking of a PIB-PU using a thermal-activated crosslinker that is part of a soft segment of a PIB-PU prepolymer according to an embodiment of the present disclosure.

FIG. 13A is a schematic diagram of an example artificial meniscus according to an embodiment of the present disclosure.

FIG. 13B1 to 13B5 illustrate exemplary process steps to fabricate the example artificial meniscus of FIG. 13A 1300 that includes a core 1301 of softer PIB-PU (possibly with crosslinks as described herein) encapsulated by a shell 1303 of harder PIB-PU (possibly with crosslinks as described herein).

FIGS. 14A and 14B are images that illustrate implantation of an exemplary disk-like synthetic polymer meniscal implant formed from PIB-PU. The synthetic meniscal implant is implanted into the space between the femur and tibia of the human knee of a patient.

FIG. 15 is a schematic diagram that illustrates implantation of an example synthetic polymer ACL implant formed from PIB-PU into a human knee.

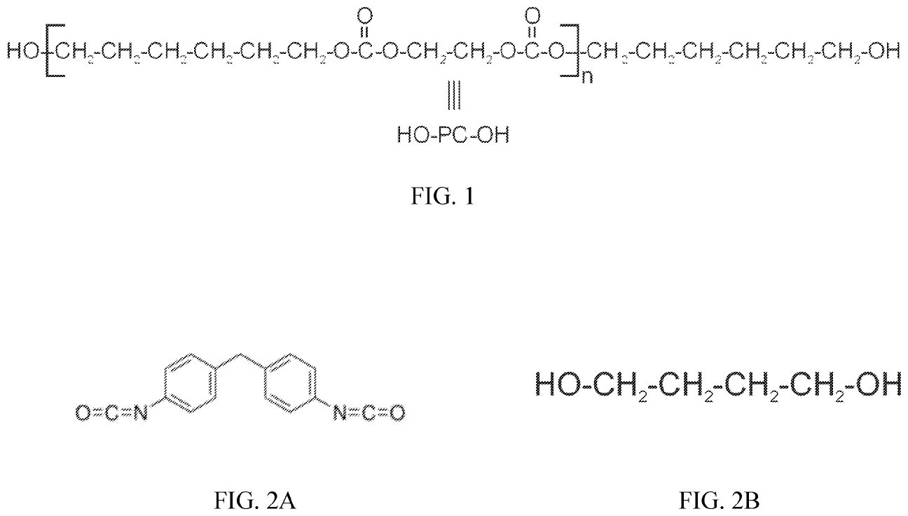

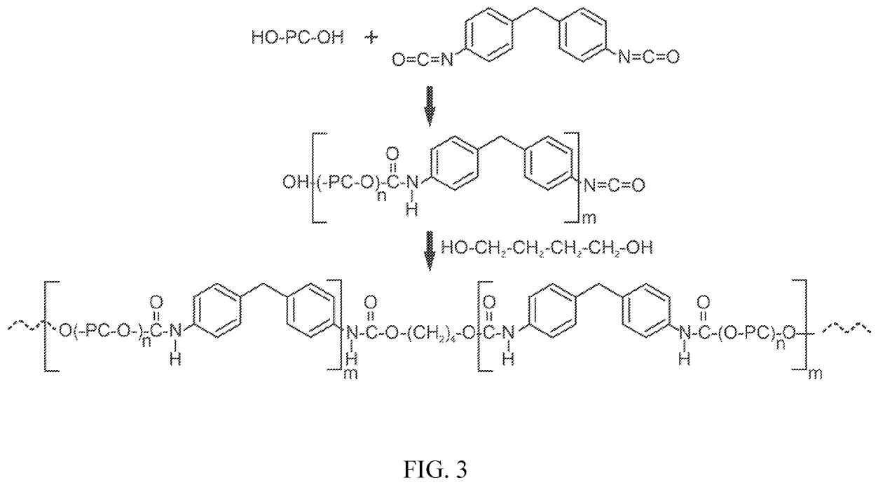

### DETAILED DESCRIPTION OF THE PREFERRED EMBODIMENTS

As used herein, the term “diol” refers to a chemical compound containing two hydroxyl groups (—OH groups).

As used herein, the term “triol” refers to a chemical compound containing three hydroxyl groups (—OH groups).

As used herein, the term “polyol” refers to a chemical compound containing multiple hydroxyl groups (—OH groups).

As used herein, the term “PIB” means polyisobutylene, which is a polymer composed of isobutylene monomers.

As used herein, the term “polyurethane” is a polymer consisting of a chain of organic units joined by urethane (carbamate, —NH—COO—) links.

As used herein, the term “polyurea” is a polymer consisting of a chain of organic units joined by urea (—NH—CO—NH—) links.

As used herein, the term “PIB-PU” means a polyisobutylene-based polyurethane. The term includes the polyisobutylene-based polyurethanes described herein. The polyisobutylene-based polyurethane is often called a polyisobutylene-urethane and is often abbreviated as “PIU”, which in this patent is the same as “PIB-PU.”

As used herein, the term “isocyanate” refers to a chemical compound having at least one isocyanate (R—N═C═O) group.

As used herein, the term “diisocyanate” refers to a chemical compound having two isocyanate (O═C═N—R—N═C═O) groups.

As used herein, the term “MDI” refers to 4,4′-methylenebis (phenyl isocyanate), wherein the “4” refers to the fourth carbon on ring one and the 4′ refers to the fourth carbon on ring 2. As there is one isocyanate on each ring, the term diisocyanate is not relevant. On the other hand, the shorthand term “MDI” is the abbreviation of Methylene DiIsocyanate.

As used herein, the term “BCB” refers to benzylcyclobutene, which is a chemical structure or functional group comprised of a benzene ring fused to a cyclobutane ring and having the chemical formula C₈H₈.

As used herein, the term “VBCB” refers to a monomer containing a styrene ring (C₈H₈) fused to a cyclobutane ring and having the summative chemical formula C₁₀H₁₀. The VBCB monomer can be reacted with a +PIB+ precursor to incorporate BCB into a polyisobutylene-based material. Once incorporated into the polyisobutylene-based material, “BCB” will be identified as the reaction site for thermal-activated crosslinking as described herein.

As used herein, the term “chain extender” refers to a lower molecular weight reagent that converts a polymeric precursor to a higher molecular weight derivative.

As used herein, the term “amine” refers to a chemical compound that contains a basic nitrogen atom with a lone pair.

Polyurethanes are generally synthesized by the reaction of a polyol, a diisocyanate and a chain extender. More specifically, the polyol is reacted with the diisocyanate to form a prepolymer, and the prepolymer is reacted with a chain extender to form the polyurethane. The polyols contribute to soft segments of polymer chains in the polyurethane, and the diisocyanate and the chain extender contribute to hard segments of polymer chains in the polyurethane. There are many types of polyols, diisocyanates, and chain extenders that can be used to form polyurethanes with varying properties.

Polyureas are generally synthesized from the reaction product of an amine-containing (soft segment) and an isocyanate.

Polyurethaneureas, or poly (urethane urea), are generally composed of polyurethane and polyurea compounds.

The physical properties of polyurethanes, polyureas, and polyurethaneureas are derived from the phase separation of the soft segment domains and hard segment domains of the polymer chain and the linkages therebetween.

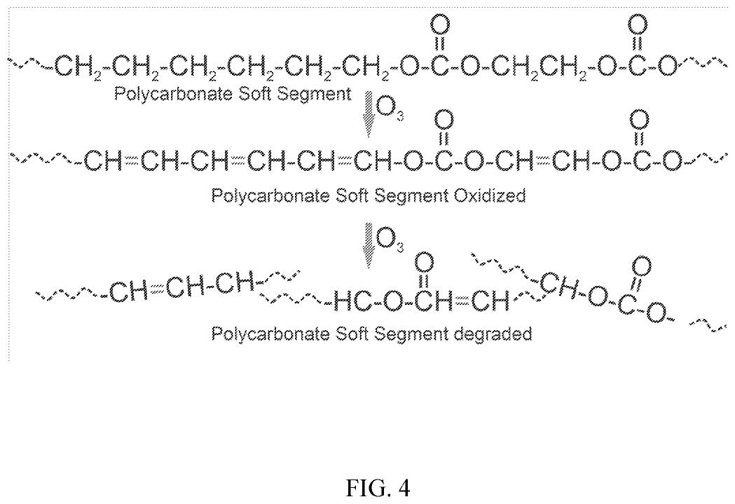

In accordance with the present invention, an orthopedic device, such as disk-like meniscal implant or an artificial ACL, is formed from polyisobutylene-polyurethane (PIB-PU). The PIB-PU is much more biostable, tougher and long-lasting than the PCU described above with respect to FIG. 4.

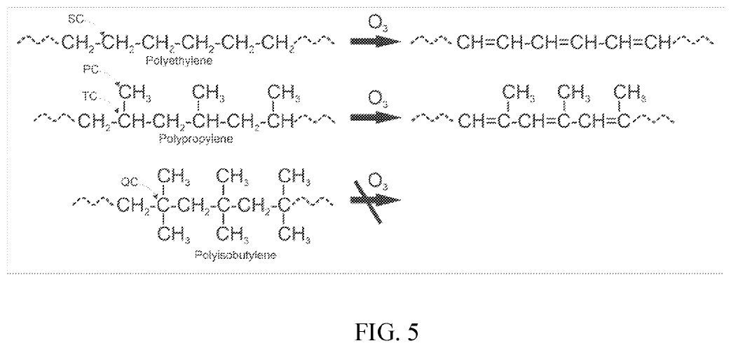

The PIB-PU of the present disclosure can be formed from a polyisobutylene-based material that has no ability to embrittle. Not to be bound by any theory, Error! Reference source not found. shows the relative stability of polyethylene versus polypropylene versus polyisobutylene. Specifically, FIG. 5 shows polyethylene with secondary carbons (SC), polypropylene with tertiary carbons (TC), and polyisobutylene with quaternary carbons (QC). It also shows oxidation by O₃ from macrophages to double bond formation. Polyisobutylene cannot form double bonds on its backbone as carbon cannot have 5 bonds, and therefore cannot oxidize. Thus, polyethylene and polypropylene can oxidize to form double bonds. Polyisobutylene cannot oxidize as the quaternary carbon will not allow double bonds to occur-the quaternary carbon would have to have 5 bonds for the reaction to go to the right.

Further, the alternating quaternary and secondary carbon backbone of polyisobutylene does not allow oxygen or moisture through as the repeating quaternary carbon backbone is very dense with little space for atoms/molecules to penetrate, and therefore there is far less chance of oxidizing or hydrolyzing the urethane or urea group.

In one embodiment, the PIB-PU of the orthopedic device can be formed by reacting a polyisobutylene-based compound (e.g., polyisobutylene diol) with a diisocyanate (e.g., MDI) to form a PIB-PU prepolymer. The PIB-PU prepolymer can be reacted with a chain extender (e.g., 1,4-butanediol) to form the resultant PIB-PU. This reaction and resulting structure are shown schematically in Error! Reference source not found.6.

In alternate embodiments, the chain extender can be ethylenediol or ethylenediamine, which can form PIB-based polyurethane ureas, perfloroethylene diols, or fluorinated versions of the PIB-polyurethane can also be used.

In yet other embodiments, the polyisobutylene-based component as described herein can be a mixture of a polyisobutylene material (e.g., polyisobutylene diol) and polytetramethylene glycol or even some of the polycarbonate diol (Error! Reference source not found.) to ensure proper polymerization as long as the dominant component of the resulting soft segments is the polyisobutylene-based material.

In embodiments, the PIB-PU of the orthopedic device can provide a tensile strength greater than 20 MPa and/or an elongation between 50% and 600%.

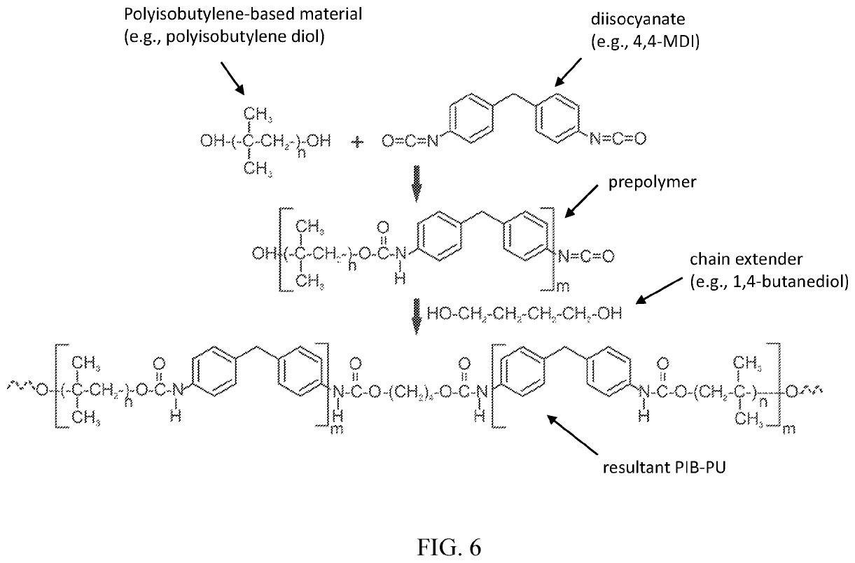

For example, in the exemplary PIB-PU of FIG. 6, elongation is controlled by i) the molecular weight of the polyisobutylene-based component and ii) the ratio of the polyisobutylene-based component to the combined weight of the diisocyanate and the chain extender. In brief, the higher the molecular weight of the polyisobutylene-based component, the higher the elongation; and the higher the ratio of the polyisobutylene-based component to the combined weight of the diisocyanate and the chain extender, the higher the elongation. For a soft polyurethane of Shore 80A, the molecular weight of the polyisobutylene-based component is typically about 2500 Daltons. The ratio of the polyisobutylene-based component to the combined weight of the diisocyanate and the chain extender can be 1:1. The elongation of the resultant PIB-PU can be about 600%. If the molecular weight of the polyisobutylene-based component is dropped to 1000 Daltons, the elongation may be approximately 200% and the hardness will be increased to approximately Shore 60D.

The tensile properties (tensile modulus, tensile strength) of the PIB-PU are somewhat fixed and dependent upon the polarities of the polyisobutylene, the diisocyanate, the chain extender, and associated hydrogen bonds and Van der Walls forces (also called hydrophobic interactions). In embodiments, the sum of the hydroxyl groups on both the polyisobutylene-based component and chain extender of the PIB-PU should be equal to or slightly less than (<0.2%) than the number of isocyanate groups of the diisocyanate (e.g., MDI). For example, the PIB-PU of FIG. 6 can be isocyanate terminated with approximately 0.2% excess isocyanate. The tensile strength of this PIB-PU can range from 15 to 30 MPa, usually 18 to 25 MPa, where the softer polyurethanes tend to have tensile strengths on the lower end of the range as compared to the harder polyurethanes which tend to be on the upper end of the range. The harder the PIB-PU, the more crystalline and the more polar groups for hydrogen bonding, the higher the tensile strength.

In embodiments, the tensile properties (tensile modulus, tensile strength) of the PIB-PU as described herein can be tailored for the particular orthopedic device. This tailoring can be achieved by varying the molecular weight of the polyisobutylene-based component as well as the ratio of the polyisobutylene-based component to the combined weight of the diisocyanate and the chain extender (the hard segment). As described herein, different molecular weights and ratios of soft segment to hard segment are synthesized and their subsequent mechanical properties (stress, strain, modulus, etc.) are measured and are compared and matched to the desired physical properties of the orthopedic device to be emulated.

In embodiments, the PIB-PU polymer of the orthopedic devices described herein can reinforced with filament made from metal (such as nitinol, titanium, stainless steel and the like) or high-molecular weight polymers (such as polyethylene, polyamide (Nylons), poly(alphamethyl)styrene, polyester terephthalate, polymethylmethacrylate, per-fluoroethylene, and the like). Similarly, the PIB-PU polymer of the orthopedic devices described herein device can be coated with non-oxidizing polymers or coatings like Poly(styrene-block-isobutylene-block-styrene), or Poly(alphamethylstyrene-block-isobutylene-block-alphamethylstyrene).

Furthermore, the PIB-PU of the orthopedic devices as described herein can be crosslinked, for example using tertiary isocyanates and/or triols. In another example, a thermal-activated crosslinker (e.g., VBCB) can be added to the polyisobutylene-based material of the soft segment (e.g., polyisobutylene diol) and once in the mold, activated to form crosslinks in the PIB-PU of the orthopedic device described herein, which is similar to the crosslinking in the polyolefin material that forms an intraocular lens as described in U.S. Pat. No. 8,765,895. The crosslinks are formed between polymer chains in the PIB-PU by covalent chemical bonds or by physical interaction between polymer chains in the PIB-PU or both. It is noteworthy that Leonard Pinchuk, a co-inventor of U.S. Pat. No. 8,765,895 is also the inventor of the present disclosure.

### EXAMPLE 1—HIGH TENSILE STRENGTH BIOSTABLE PIB-PU THAT IS CROSSLINKED BETWEEN HARD SEGMENTS

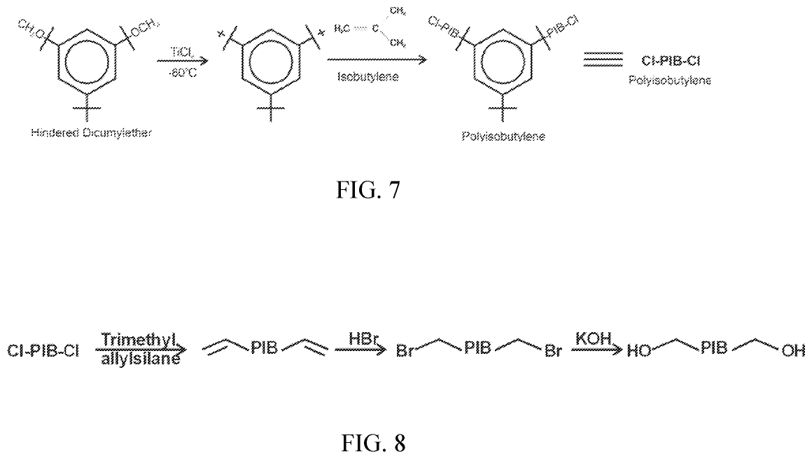

In this Example 1, the first step is to form polyisobutylene using carbo-cationic polymerization chemistry well documented by Kennedy et al. for example, see J. P. Kennedy, J. E. Puskas, G. Kaszas, W. G. Hager, “Thermoplastic elastomers of isobutylene and process of preparation, U.S. Pat. No. 4,946,899 (1990); and Wang B, Mishra M K, Kennedy J P, “Living carbocationic polymerization XII. Telechelic polyisobutylenes by a sterically hindered bifunctional initiator,” Polym Bull 1987; 17:205-11. An example of this reaction is shown in FIG. 7, which forms a di-cation from a seed molecule (hindered dicumylether) and a Lewis acid (TiCl₄). The di-cation is reacted with isobutylene gas to form polyisobutylene. As the resultant polymer will have a molecular weight between 1,000 and 10,000 Daltons, with the majority of the polymer being PIB, it is reasonable to represent the reaction product as CI-PIB-CI shown on the far right. The Cl− groups on the ends of the PIB are from the TiCl4 and provide the counter-ions for the cations.

In the second step of Example 1, the polyisobutylene (Cl-PIB-Cl) is converted to a hydroxyl-terminated polyisobutylene diol using the procedures described by Kennedy et al. in U.S. Pat. No. 9,587,067 as shown in FIG. 8.

In other embodiments, the hydroxyl-terminated polyisobutylene can include a mixture of the polyisobutylene diol and polytetramethylene glycol or a polycarbonate diol.

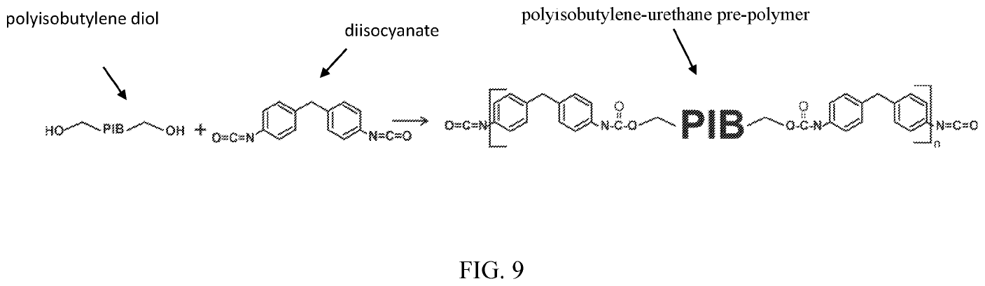

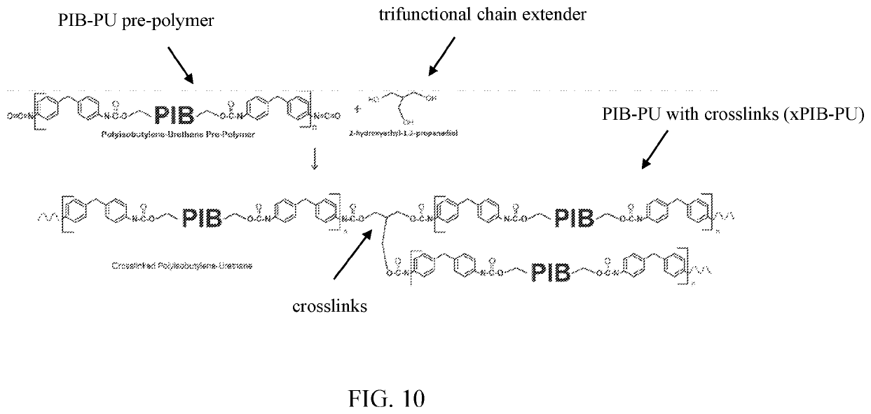

In the third step of Example 1, the hydroxyl-terminated polyisobutylene is reacted with an excess of diisocyanate (such as MDI) to form a PIB-PU pre-polymer as shown in FIG. 9 Note that this PIB-PU pre-polymer can be a linear polyisobutylene-urethane pre-polymer with predominantly PIB as exemplified by the larger PIB in the schematic of Error! Reference source not found.

In embodiments, the diisocyanate used to form the PIB-PU prepolymer can be 4,4′-Methylenebis(phenyl isocyanate), 4,4′-Methylenebis(cyclohexane isocyanate) (also called hydrogenated MDI), 2,4-toluene diisocyanate, or 1,6-hexamethylene diisocyanate, and the like.

In the fourth step of Example 1, the PIB-PU pre-polymer is reacted with a trifunctional chain extender (e.g., 2-hydroxyethyl-1,3-propanediol) in the presence of heat to yield a PIB-PU with crosslinks between hard segments of the polymer chains of the PIB-PU as shown in Error! Reference source not found. Note that the functionality of a compound relates to the presence of functional groups in the compound. For example, a monofunctional compound possesses one functional group, a difunctional compound possesses two functional groups, a trifunctional compound possesses three functional groups, and so forth. The crosslinks are derived from the reaction of the trifunctional chain extender and the isocyanate groups of the diisocyanate.

In embodiments, the trifunctional chain extender can be 2-hydroxyethyl-1,3-propanediol, 1,2,3-Propanetriol (glycerin), 1,2,3-propanetriamine, 2-hydroxyethyl-1,4-butanediol, 2-hydroxypropyl-1,4-butanediol, 3-hydroxypropyl-1,5-pentanediol, 3-aminopropyl-1,5-pentanediamine, 4-hydroxybutyl-1,6-hexanediol, 3-hydroxybutyl-1,6-hexanediol, and the like or combinations thereof.

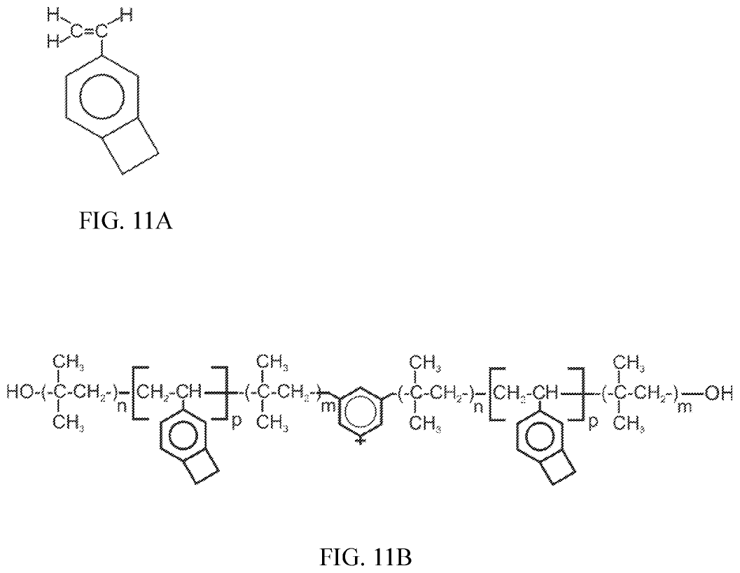

Note that the trifunctional chain extender of 2-hydroxyethyl-1,3-propanediol includes three primary hydroxyl groups (—CH₂—OH groups) as shown in FIG. 10.

### EXAMPLE 2—HIGH TENSILE STRENGTH BIOSTABLE PIB-PU THAT IS CROSSLINKED BETWEEN SOFT SEGMENTS

In this Example 2, the first step is to form polyisobutylene that includes a thermal-activated crosslinker. For example, the thermal-activated crosslinker can be BCB derived a suitable monomer such as 4-vinylbenzocyclobutene (VBCB) (FIGS. 11A), 4-methylvinylbenzocyclobutene, 4-vinylbenzocyclopropene, 4-vinylbenzo-2-methylcyclobutene, 4-vinylbenzo-2-ethylcyclobutene or combinations thereof can be integrated into the PIB diol. In embodiments, the VBCB or other suitable monomer can be added neat or diluted in a solvent (e.g., methylcyclohexane) and added to the growing PIB chain formed by carbo-cationic polymerization. The VBCB or other suitable monomer can be added to carbo-cationic polymerization reaction at random or predefined time intervals.

In one embodiment, VBCB or other suitable monomer can be added to the carbo-cationic polymerization reaction in the middle of the growing PIB chain. So, when half of the isobutylene gas has been added, VBCB or other suitable monomer, at an equivalent molar level of 1 to 5 times (preferably 1) the molar content of the dicumylether initiator, can be added to the reaction mixture. After 1 to 10 minutes (time for the VBCB or other suitable monomer to incorporate into the chain), the remaining isobutylene gas can be added in the usual manner. In this manner, the VBCB or other suitable monomer can be added halfway along the polyisobutylene chain to yield Cl-PIB-BCB-PIB-Cl and thus integrate BCB into the PIB diol. If only one crosslinking site is desired to one PIB, the VBCB or other suitable monomer can be added at the same molar concentration as the dicumylether initiator.

In the second step of Example 2, the resultant PIB is converted to a hydroxyl-terminated polyisobutylene diol with thermal-activated crosslinker (e.g., OH-PIB-BCB-PIB-OH) using the procedures described by Kennedy et al. in U.S. Pat. No. 9,587,067. An example of the hydroxyl-terminated polyisobutylene diol with thermal-activated crosslinker is shown in FIG. 11B. This conversion can be performed at temperatures much lower than the temperatures required to activate the crosslinking of the thermal-activated crosslinker (e.g., BCB).

In other embodiments, the hydroxyl-terminated polyisobutylene can include a mixture of the polyisobutylene diol (with thermal-activated crosslinker (e.g., OH-PIB-BCB-PIB-OH)) and polytetramethylene glycol or a polycarbonate diol or PIB diol.

In the third step of Example 2, the hydroxyl-terminated polyisobutylene with thermal-activated crosslinker is reacted with an excess of diisocyanate (e.g., MDI) to form a PIB-PU pre-polymer with a thermal-activated crosslinker (e.g., BCB) in the soft segments of the prepolymer. This reaction can be performed at temperatures much lower than the temperatures required to activate the crosslinking of the thermal-activated crosslinker (e.g., BCB).

In embodiments, the diisocyanate used to form the PIB-PU prepolymer can be 4,4′-methylenebis (phenyl isocyanate) (MDI), 4,4′-methylenebis(cyclohexane isocyanate) (also called hydrogenated MDI), 2,4-toluene diisocyanate, or 1,6-hexamethylene diisocyanate, and the like. In embodiments, the diisocyanate (e.g., MDI) can be added slowly to the hydroxyl-terminated polyisobutylene diol with thermal-activated crosslinker in order to allow the exotherm to dissipate, or one can cool the reaction with a cold water blanket. Furthermore, a catalyst can be used in this reaction. For example, the catalyst can include dibutyl tin dilaurate (DBTDL) (≈0.1 to 0.2% by weight of solids) or stannous octoate (≈0.5 to 2% by weight of solids).

In an optional fourth step of Example 2, the PIB-PU pre-polymer with a thermal-activated crosslinker can be reacted with a chain extender to consume excess diisocyanate and link the remaining prepolymer strands together. This reaction can be done neat or in a solvent. In this reaction, the total number of functional groups that terminate the PIB-PU pre-polymer with a thermal-activated crosslinker should equal the total number of hydroxyl groups from the sum of the soft segment and chain extender.

In embodiments, the chain extender can be ethylenediamine, ethylenediol, propylenediamine, propylenediol, 1,4-butnediol, 1,4-butanediamine, 1,6-hexanediol, and larger molecules with secondary hydroxyl or amine groups.

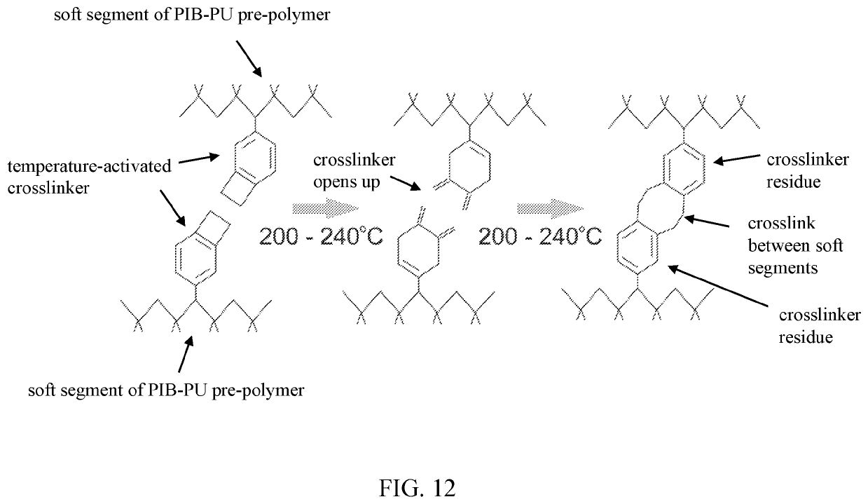

In the fifth step of Example 2, heat is applied to PIB-PU pre-polymer that results from the fourth step (or the third step if the fourth step is omitted) to form crosslinks between soft segments of the polymer chains of the PIB-PU. The crosslinks between the soft segments of the polymer chains of the PIB-PU are formed by temperature activation of the crosslinker that is integrated into the soft segments of the PIB-PU pre-polymer. For example, in the case that BCB is used as the thermal-activated crosslinker, at high temperatures in the range of 200-240° C., the strained cyclobutene ring of the BCB opens up and rearranges to form crosslinks between soft segments of the polymer chains of the resultant PIB-PU polymer with residues of the crosslinker as shown in FIG. 12. The thermal-crosslinking of the PIB-PU can be performed in the specific mold to provide the product used for the implantable application.

### EXAMPLE 3—HIGH TENSILE STRENGTH BIOSTABLE PIB-PU THAT IS CROSSLINKED BETWEEN BOTH SOFT SEGMENTS AND HARD SEGMENTS

In this Example 3, the first three steps of Example 2 are performed to form a PIB-PU pre-polymer with a thermal-activated crosslinker (e.g., BCB) in the soft segments of the prepolymer.

In the fourth step of Example 3, the PIB-PU prepolymer with thermal-activated crosslinker (e.g., BCB) in the soft segments of the prepolymer is reacted with a trifunctional chain extender (e.g., 2-hydroxyethyl-1,3-propanediol) in the presence of heat to yield a PIB-PU with crosslinks between hard segments derived from the diisocyanate. Prior to the reaction, any solvents can be flashed off under vacuum at, say, 100° C.

In embodiments, the trifunctional chain extender can be 2-hydroxyethyl-1,3-propanediol, 1,2,3-Propanetriol (glycerin), 1,2,3-propanetriamine, 2-hydroxyethyl-1,4-butanediol, 2-hydroxypropyl-1,4-butanediol, 3-hydroxypropyl-1,5-pentanediol, 3-aminopropyl-1,5-pentanediamine, 4-hydroxybutyl-1,6-hexanediol, 3-hydroxybutyl-1,6-Hexanediol, and the like or combinations thereof.

In the fifth step of Example 3, heat is applied to PIB-PU pre-polymer that results from the fourth step to form crosslinks in the soft segments of the PIB-PU. For example, in the case that BCB is used as the thermal-activated crosslinker, at high temperatures in the range of 200-240° C., the strained cyclobutene ring of the BCB opens up and rearranges to form crosslinks in the soft segment of the resultant PIB-PU polymer with residues of the crosslinker as shown in FIG. 12.

The combination of the fourth and fifth steps forms crosslinks in both the soft segment and the hard segment of the resultant PIB-PU polymer.

### Advantages of PIB-PU Polymer with Crosslinks

PIB-PU with crosslinks as described herein offer several advantages over non-crosslinked polymers as summarized below.

First, PIB-PU with crosslinks can have enhanced mechanical strength and stability compared to non-crosslinked polymers. The chemical crosslinking creates a three-dimensional network of covalent bonds, which makes the polymer more rigid, resistant to deformation, and able to withstand higher mechanical stresses. This property is beneficial in applications that require durability, load-bearing capacity, and structural integrity, such as the medical devices described herein.

Second, PIB-PU with crosslinks can exhibit improved dimensional stability. The crosslinks restrict molecular movement, reducing the tendency of the polymer to expand or contract in response to temperature changes or external forces. This stability is crucial in applications where maintaining precise dimensions is essential, such as the medical devices described herein.

Third, PIB-PU with crosslinks can possess lower swelling and solubility compared to non-crosslinked polymers. The crosslinks create a tighter network, reducing the ability of solvents or liquids to penetrate and swell and weaken the polymer structure. This property is advantageous in applications where chemical resistance, dimensional stability, and resistance to fluid absorption are critical, such as in the medical devices described herein.

Fourth, PIB-PU with crosslinks can offer improved durability and resistance to wear and tear. The interconnected crosslinks provide a stronger network that can withstand repeated mechanical stresses, impact, and abrasion. This property can minimize creep deformation and improves abrasion resistance over time and makes the crosslinked polymer suitable for applications that require long-term durability, such as in the medical devices described herein.

Fifth, PIB-PU with crosslinks can have excellent shape retention, also called creep deformation resistance. Once formed, the crosslinked structure retains its shape and prevents the polymer from flowing or deforming under normal operating conditions. This property is beneficial in applications where maintaining precise shapes and geometries is crucial, such as in the medical devices described herein.

Overall, PIB-PU with crosslinks can enhance their mechanical, thermal, chemical, electrical, and dimensional properties, providing improved performance and expanding their range of applications compared to non-crosslinked polymers.

In embodiments, PIB-PU can form an orthopedic device such as an artificial meniscus or ACL. PIB-PU can possibly form other artificial orthopedic implants, such as rotator cuff labrums, spinal disks, finger joints, impact dampening liners for the artificial hip and knee prosthesis, and a soft tissue replacement, such as heart valve leaflets, vascular grafts, and more.

In other embodiments, an article of manufacture is provided that is formed from PIB-PU. The article of manufacture can be a medical implant device, such as a synthetic heart valve used in Trans Aortic Valve Replacement (TAVR), a vascular graft, a cardiac pacemaker lead, a defibrillator lead, a catheter, an implantable prosthesis, a cardiac assist device, an artificial organ, and a drug delivery device.

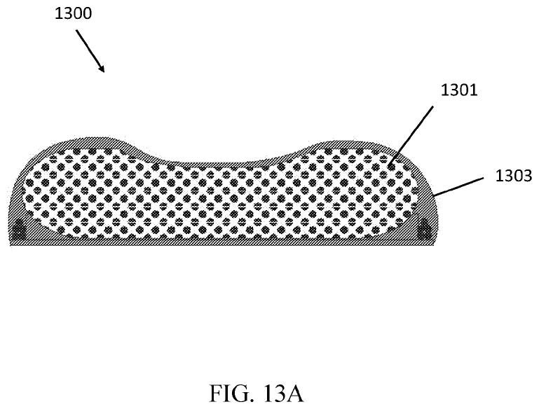

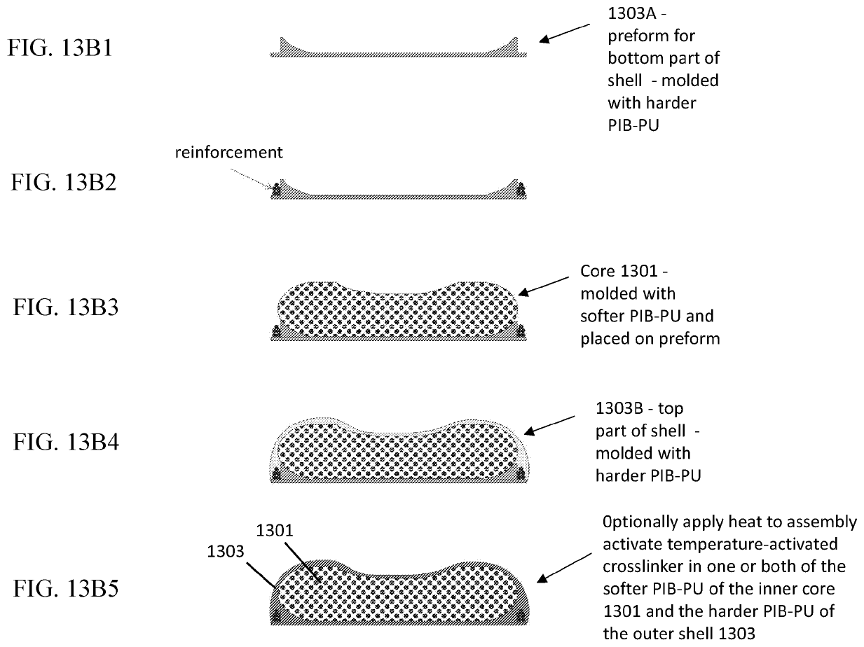

FIG. 13A illustrates an example artificial meniscus 1300 that includes a core 1301 of softer PIB-PU (possibly with crosslinks as described herein) encapsulated by a shell 1303 of harder PIB-PU (possibly with crosslinks as described herein).

In embodiments shown in FIG. 13B1 to 13B5, a bottom part 1303A of the outer shell of harder PIB-PU can be molded to form a preform that provides a scaffold for the softer PIB-PU core 1301 and reinforcement. See FIG. 13B2. In embodiments, the reinforcement can be filament or structures made from metal (such as nitinol, titanium, stainless steel, cobalt-chromium-nickel, and the like) and/or high-molecular weight polymers (such as polyethylene, polyamide (Nylons), poly(alphamethyl)styrene, polyester terephthalate, polymethylmethacrylate, per-fluoroethylene, and the like). The softer PIB-PU core 1301 can be molded and then placed on the preform as shown in FIG. 13B3. A top part 1303B of the outer shell of harder PIB-PU can be molded around the softer PIB-PU core 1301 such that the bottom part 1303A and the top part 1303B encapsulate the softer PIB-PU core 1301 as shown in FIG. 13B4. For the case that the PIB-PU of the shell 1303 and/or the core 1301 incorporate a thermal-activated crosslinker, the resultant assembly can be subject to heat to activate the thermal-activated crosslinker to form the crosslinks in the soft segment of the PIB-PU as shown in FIG. 13B5. This can help adhere the core 1301 to the shell 1303 for enhanced structural integrity of the artificial meniscus 1300. Furthermore, the resultant assembly can be coated with non-oxidizing polymers or coatings like Poly(styrene-block-isobutylene-block-styrene), or Poly(alphamethylstyrene-block-isobutylene-block-alphamethylstyrene).

In embodiments, the softer PIB-PU of the inner core 1301 can be configured to provide desired shock absorption, and the harder PIB-PU of the outer shell 1303 can be configured to provide desired ware resistance.

In embodiments, the softer PIB-PU of the inner core 1301 can have a molecular weight of approximately 2000 to 5000 Daltons with the weight percent of PIB-based components of about 65% to 75% of the total weight of the PIB-PU. The Shore Hardness of the PIB-PU of the core 1301 can be about Shore 70A to 80A, and the tensile strength will be approximately 20-30 MPa with an elongation greater than 400%.

In embodiments, the harder PIB-PU of the outer shell 1303 can have a molecular weight of 750 to 1500 Daltons with the weight percent of PIB-based components of about 40% to 60% of the total weight of the PIB-PU. The Shore Hardness of the PIB-PU of the shell 1303 can be about Shore 55D to 75D, and the tensile strength will be approximately 25-35 MPa with an elongation less than 200%.

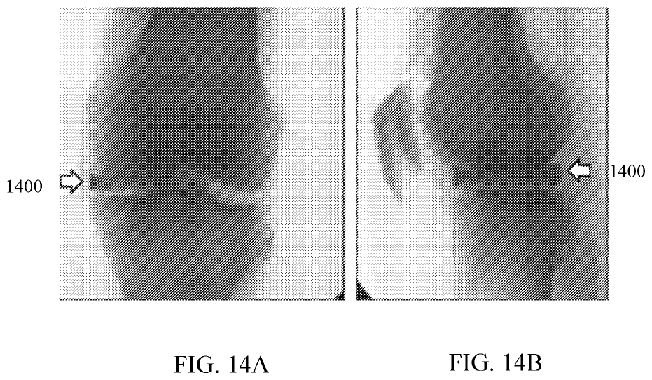

FIGS. 14A and 14B illustrate the implantation of disk-like synthetic polymer meniscal implant 1400 formed from PIB-PU. The synthetic meniscal implant 1400 is implanted into the space between the femur and tibia of the human knee of a patient as pointed to by the arrows in FIGS. 14A and 14B. In embodiments, the implant 1400 can embody the artificial meniscus 1300 described above with respect to FIG. 13A and FIG. 13B1 to 13B5.

In embodiments, the tensile properties (tensile modulus, tensile strength, elongation) of the PIB-PU as described herein can be tailored to match a desired modulus value of the normal knee meniscus. This matching can be achieved by varying the molecular weight of the PIB-based component as well as the ratio of PIB-based component to hard segment components in different samples of PIB-PU formed as described herein. The tensile properties for various samples are measured and matched to the required moduli of the cartilage or ligament to be replaced.

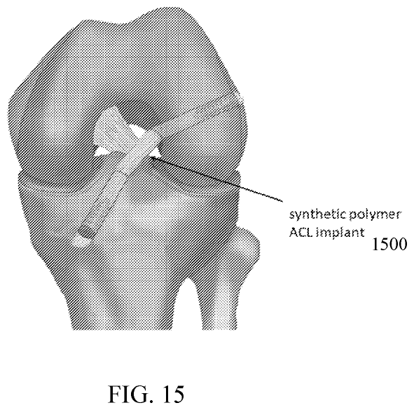

FIG. 15 illustrates the implantation of synthetic polymer ACL implant 1500 formed from PIB-PU into a human knee. The artificial ACL implant can be implanted into the human knee of a patent using the well-known ligament augmentation and reconstruction system (LARS). Like the meniscus, the ACL can be reinforced with metal or polymeric fibers to limit its extension and creep deformation.

In embodiments, the article of manufactures described herein can be formed from PIB-PU using injection or compression molding, extrusion, spinning, or other suitable method(s).

In embodiments, one or more of the reactions and process steps as described herein can be carried out in a mold that is used to shape and form the article of manufacture.

In one embodiment, the reaction of the fourth step of Example 1 can be carried out in a mold that is shaped to form the article of manufacture. For example, softer PIB-PU can be molded to form the inner core 1301 of the artificial meniscus of FIG. 13A where the reaction of the fourth step of Example 1 is carried out in a mold that is shaped to form the inner core 1301. Similarly, the reaction of the fourth step of Example 1 can be carried out in separate molds that are shaped to form the bottom part 1303A and top part 1303B of the outer shell of the meniscus.

In another embodiment, the reaction of the optional fourth step and the heating of the fifth step of Example 2 can be carried out in a mold that is shaped to form the article of manufacture. For example, softer PIB-PU can be molded to form the inner core 1301 of the artificial meniscus of FIG. 13A where the reaction of the optional fourth step and the heating of the fifth step of Example 2 are carried out in a mold that is shaped to form the inner core 1301. Similarly, the reaction of the optional fourth step and the heating of the fifth step of Example 2 can be carried out in separate molds that are shaped to form the bottom part 1303A and top part 1303B of the outer shell of the meniscus.

In yet another embodiment, the reaction of the fourth step and the heating of the fifth step of Example 3 can be carried out in a mold that is shaped to form the article of manufacture. For example, softer PIB-PU can be molded to form the inner core 1301 of the artificial meniscus of FIG. 13A where the reaction of the fourth step and the heating of the fifth step of Example 3 are carried out in a mold that is shaped to form the inner core 1301. Similarly, the reaction of the fourth step and the heating of the fifth step of Example 3 can be carried out in separate molds that are shaped to form the bottom part 1303A and top part 1303B of the outer shell of the meniscus.

While particular embodiments have been shown and described, it will be understood by those skilled in the art that various changes in form and details may be made without deviating from the spirit and scope of the invention encompassed by the appended claims.

1. A medical implant device comprising:
a polyurethane or polyurea polymer including hard segments and soft segments with crosslinks between polymer chains in the polymer, wherein the hard segments include at least one of a urethane, urea, or urethane urea derived from a diisocyanate, wherein the soft segments include polyisobutylene derived from hydroxyl-terminated polyisobutylene;
wherein the crosslinks between polymer chains in the polymer include first crosslinks that link to the hard segments of the polymer chains and second crosslinks that link to the soft segments of the polymer chains, wherein the first crosslinks include parts of a chain extender linked to the hard segments, and wherein the second crosslinks include parts of a thermally-activated crosslinker linked to the soft segments.

2-5. (canceled)

6. A medical implant device according to claim 1, wherein:
the chain extender of the first crosslinks comprises a trifunctional chain extender.

7. A medical implant device according to claim 6, wherein:
the trifunctional chain extender is selected from the group consisting of 2-hydroxyethyl-1,3-propanediol, 1,2,3-Propanetriol (glycerin), 1,2,3-propanetriamine, 2-hydroxyethyl-1,4-butanediol, 2-hydroxypropyl-1,4-butanediol, 3-hydroxypropyl-1,5-pentanediol, 3-aminopropyl-1,5-pentanediamine, 4-hydroxybutyl-1,6-hexanediol, 3-hydroxybutyl-1,6-hexanediol, or combinations thereof.

8. (canceled)

9. A medical implant device according to claim 1, wherein:
the thermal-activated crosslinker of the second crosslinks comprises BCB, which is preferably derived from a compound selected from the group consisting of 4-vinylbenzocyclobutene (VBCB), 4-methylvinylbenzocyclobutene, 4-vinylbenzocyclopropene, 4-vinylbenzo-2-methylcyclobutene, 4-vinylbenzo-2-ethylcyclobutene, or combinations thereof.

10. A medical implant device according to claim 1, wherein:
the hydroxyl-terminated polyisobutylene comprises hydroxyl-terminated polyisobutylene diol.

11. A medical implant device according to claim 1, wherein:
the hydroxyl-terminated polyisobutylene comprises hydroxyl-terminated polyisobutylene diol and at least one of polytetramethylene glycol and a polycarbonate diol.

12. A medical implant device according to claim 1, wherein:
the diisocyanate comprises MDI.

13. A medical implant device according to claim 1, wherein the medical implant device is an orthopedic implant device selected from the group consisting of an artificial meniscus, ACL, rotator cuff labrum, spinal disk, finger joint, impact dampening liner for artificial hip or knee prosthesis, and a soft tissue replacement.

14. A medical implant device according to claim 1, wherein the medical implant device is selected from the group consisting of a synthetic heart valve, a vascular graft, a cardiac pacemaker lead, a defibrillator lead, a catheter, an implantable prosthesis, a cardiac assist device, an artificial organ, and a drug delivery device.

15-18. (canceled)

19. A method of preparing a polyurethane or polyurea polymer, the method comprising:
synthesizing or obtaining hydroxyl-terminated polyisobutylene that includes a thermal-activated crosslinker;
reacting the hydroxyl-terminated polyisobutylene with a diisocyanate to form a prepolymer;
reacting the prepolymer with a chain extender to form a polyurethane or polyurea polymer that includes hard segments and soft segments, wherein the hard segments include at least one of a urethane, urea, or urethane urea derived from the diisocyanate, wherein the soft segments include polyisobutylene derived from the hydroxyl-terminated polyisobutylene, and wherein the a polyurethane or polyurea polymer includes first crosslinks that link to the hard segments, wherein the first crosslinks include parts of the chain extender linked to the hard segments; and
applying heat to the polyurethane or polyurea polymer derived from the reaction of prepolymer and the chain extender to form second crosslinks that link to the soft segments of the polyurethane or polyurea polymer, wherein the second crosslinks include parts of the thermally-activated crosslinker linked to the soft segments.

20. A method according to claim 19, wherein:
the thermal-activated crosslinker comprises BCB, which is preferably derived from a compound selected from the group consisting of 4-vinylbenzocyclobutene (VBCB), 4-methylvinylbenzocyclobutene, 4-vinylbenzocyclopropene, 4-vinylbenzo-2-methylcyclobutene, 4-vinylbenzo-2-ethylcyclobutene, or combinations thereof.

21. A method that forms a medical implant device from the polyurethane or polyurea polymer of claim 19.

22. A method according to claim 21, wherein:
both the reaction of the prepolymer with the chain extender and the application of the heat is carried out in a mold used to shape and form the medical implant device.

23. A method according to claim 19, wherein:
the chain extender comprises a trifunctional chain extender.

24. A method according to claim 23, wherein:
the trifunctional chain extender is selected from the group consisting of 2-hydroxyethyl-1,3-propanediol, 1,2,3-Propanetriol (glycerin), 1,2,3-propanetriamine, 2-hydroxyethyl-1,4-butanediol, 2-hydroxypropyl-1,4-butanediol, 3-hydroxypropyl-1,5-pentanediol, 3-aminopropyl-1,5-pentanediamine, 4-hydroxybutyl-1,6-hexanediol, 3-hydroxybutyl-1,6-hexanediol,, or combinations thereof.

25. A method that forms a medical implant device from the polyurethane or polyurea polymer of claim 23.

26. A method according to claim 25, wherein:
both the reaction of the prepolymer with the trifunctional chain extender and the application of the heat is carried out in a mold used to shape and form the medical implant device.

27. An artificial meniscus comprising:
an inner core encapsulated by an outer shell,
wherein the inner core is formed from a first polyurethane or polyurea polymer including hard segments and soft segments, wherein the hard segments include at least one of a urethane, urea, or urethane urea derived from a diisocyanate, wherein the soft segments include polyisobutylene derived from hydroxyl-terminated polyisobutylene;
wherein the outer shell is formed from a second polyurethane or polyurea polymer including hard segments and soft segments, wherein the hard segments include at least one of a urethane, urea, or urethane urea derived from a diisocyanate, wherein the soft segments include polyisobutylene derived from hydroxyl-terminated polyisobutylene;
wherein the first polyurethane or polyurea polymer of the inner core is softer than the second polyurethane or polyurea polymer of the outer shell; and
wherein at least one of the first polyurethane or polyurea polymer of the inner core and the second polyurethane or polyurea polymer of the outer shell includes crosslinks between polymer chains, wherein the crosslinks between polymer chains include first crosslinks that link to the hard segments of the polymer chains and second crosslinks that link to the soft segments of the polymer chains, wherein the first crosslinks include parts of a chain extender linked to the hard segments, and wherein the second crosslinks include parts of a thermally-activated crosslinker linked to the soft segments.

28-29. (canceled)

30. An artificial meniscus according to claim 27, wherein:
the chain extender comprises a trifunctional chain extender.

31. An artificial meniscus according to claim 30, wherein:
the trifunctional chain extender is selected from the group consisting of 2-hydroxyethyl-1,3-propanediol, 1,2,3-Propanetriol (glycerin), 1,2,3-propanetriamine, 2-hydroxyethyl-1,4-butanediol, 2-hydroxypropyl-1,4-butanediol, 3-hydroxypropyl-1,5-pentanediol, 3-aminopropyl-1,5-pentanediamine, 4-hydroxybutyl-1,6-hexanediol, 3-hydroxybutyl-1,6-hexanediol, or combinations thereof.

32. (canceled)

33. An artificial meniscus according to claim 27, wherein:
the thermal-activated crosslinker comprises BCB, which is preferably derived from a compound selected from the group consisting of 4-vinylbenzocyclobutene (VBCB), 4-methylvinylbenzocyclobutene, 4-vinylbenzocyclopropene, 4-vinylbenzo-2-methylcyclobutene, 4-vinylbenzo-2-ethylcyclobutene, or combinations thereof.
